(19) United States
(12) Patent Application Publication
(10) Pub. No.: US 2021/0338970 A1
(43) Pub. Date: Nov. 4, 2021

(54) DEVICE AND METHOD FOR INCREASING THE WELL-BEING OF LIVING BEINGS

(71) Applicant: Casada Group GmbH, Paderborn (DE)

(72) Inventor: Jakob Radtke, Paderborn (DE)

(21) Appl. No.: 17/288,086

(22) PCT Filed: Oct. 16, 2019
(86) PCT No.: PCT/DE2019/100896

§ 371 Date: Apr. 23, 2021

(30) Foreign Application Priority Data
Oct. 26, 2018 (DE) 10 2018 126 800.9
Nov. 12, 2018 (DE) 10 2018 128 168.4

(51) Int. Cl. A61M 21/02; A61H 37/00

(52) U.S. Cl. CPC A61M 21/02; A61H 37/00; A61H 2201/10; A61M 2021/0022; A61H 2201/5046; A61H 2201/0149; A61H 2201/501

(57) ABSTRACT

A device for increasing the well-being of living beings, including a brain stimulator for generating a stimulation signal in the infrasonic range, the brain stimulator being used to stimulate the brain of the living being. A massager is provided for generating a massage signal, by means of which body parts of the living being can be massaged.

The invention relates to a device for increasing the well-being of living beings, comprising a brain stimulator for generating a stimulation signal in the infrasonic range, said brain stimulator being used to stimulate the brain of the living being.

The invention further relates to a method for increasing the well-being of a living being in a relaxation state, wherein a brain of the living being is stimulated with a periodical stimulation signal in an infrasonic range.

A device for promoting restful sleep is known from EP 2 069 001 A1. To this end, a brain stimulator is used which generates a stimulation signal in the infrasonic range, said stimulation signal being induced in the brain of the person via electrodes attached to a head of said person. EEG-Signals are detected in the brain which are concentrated in a frequency range of 0.5 Hz to 4 Hz. Alternatively, the stimulation signal can also be induced in the brain via headphones. The known device is restricted to the use for sleep disorders. It is desirable to also reduce stress during non-sleeping times in order to improve the psychological and physical well-being of a living being.

It is therefore the object of the present invention to propose a device and a method for increasing the well-being of living beings with which the extent of the relaxation state can be effectively increased or the relaxation state can be entered faster.

In order to achieve this object, the invention, in combination with the preamble of Patent claim 1, is characterized in that a massager is provided for generating a massage signal by means of which body parts of the living being can be massaged.

The particular advantage of the invention is that, due to the combined use of brain stimulation on the one hand and massage of body parts of the living being on the other hand, the effectiveness of the relaxation can be substantially improved. Studies have shown that the effectiveness of the relaxation can be increased by up to 80% in this way. The basic idea of the invention is to affect the living being, on the one hand psychologically by stimulating the brain and on the other hand physically by massage, such that, due to the combined use thereof, an improved relaxation state of the living being can be achieved which exceeds the aggregated effectiveness of brain stimulation and massage. Studies have shown that the desired relaxation state sets in significantly faster and more permanently than if the brain stimulation and the massage are staggeredly performed.

According to a preferred embodiment of the invention, an actuator is provided, said actuator being used to generate the stimulation actuation signal for actuating the brain and the massage signal for actuating the massager. This actuator is preferably integrated in a mobile data apparatus, in particular in a mobile phone, so that the user can influence the two apparatuses, i.e. the brain stimulator and the massager, in an operator-friendly manner using a relaxation application.

According to a preferred embodiment of the invention, the actuator has several relaxation programs, so that the user can choose relaxation programs with different intensities and/or lengths themselves based on the current state of the user in order to achieve the desired relaxation state at the end of the process.

According to a further development of the invention, the stimulation signal emitted by the brain stimulator is formed as a binaural beat having a preferably pulsating beat frequency, wherein different carrier frequencies are induced at differing ears of the living being. The difference between the carrier frequencies is in the low-frequency infrasonic range. The hearing system of the living being does not register the different vibrations of the stimulation signal. Instead, the interference process is transferred to the brain, wherein the ear of the living being is circumvented. Preferably, synchronization of at least two rhythm cycles of the stimulation signal is carried out.

According to a further development of the invention, the stimulation signal is overlaid with a music signal, so that an additional relaxing effect is applied to the living being through the music.

According to a further development of the invention, the brain stimulator is actuated such that, in a first time interval, the beat frequency is decreased in a range between 14 Hz and 30 Hz to a range of 7 Hz to 14 Hz or 3 Hz to 7 Hz. In this case, the living being is in a relaxation phase. In a subsequent second time interval, the living being is in a use phase in which the beat frequency reaches its lowest point. In a third time interval, a recovery phase follows in which the beat frequency increases again in the range of the original frequency. If the relaxation program is to be used for falling asleep, low stimulation frequency can be generated in the third time interval. The first time interval, the second time interval, and the third time interval can be in a range between 5 min and 10 min.

According to a further development of the invention, the massager is formed as a massage chair having means for back massaging. With a delicate back massage, the gentle recovery of under-served intervertebral discs can be initiated. The easing of emotional and physical tension is thus promoted.

In order to achieve the object, the method according to the invention, in combination with the preamble of Patent claim 11, is characterized in that, simultaneously, a body of the living being is at least partially massaged using massage means.

The particular advantage of the method according to the invention is that, due to the simultaneous use of brain stimulation and massage, in particular work-related stress can be reduced. Both the psychological and the physical well-being of a person can be substantially improved, for example, by use during a lunch break.

In the following, exemplary embodiments of the invention are described in more detail using the drawings.

Therein:

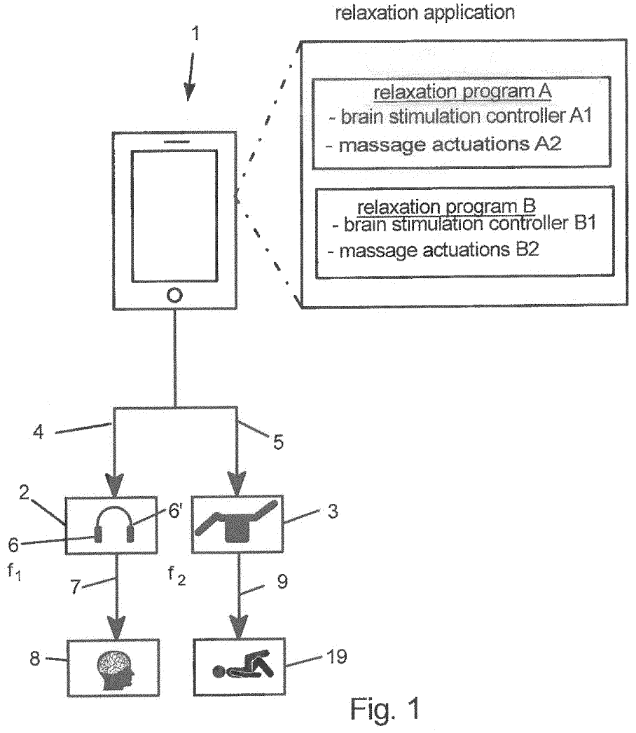

FIG. 1 shows a schematic representation of a device according to the invention,

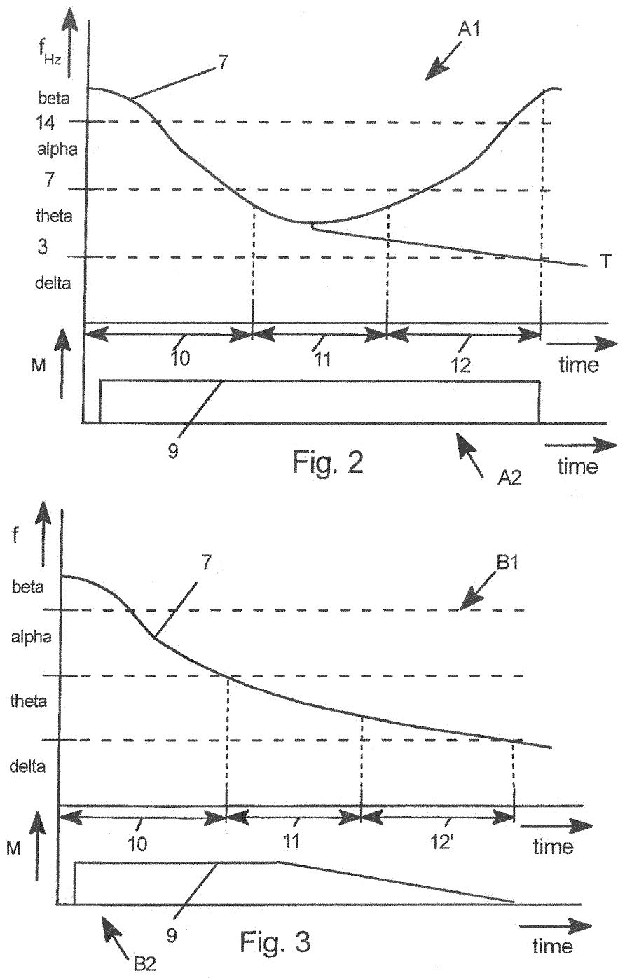

FIG. 2 shows a frequency-time chart in accordance with a first relaxation program, and

FIG. 3 shows a frequency-time chart in accordance with a second relaxation program.

A device forpsycho-physiological relaxation of living beings is preferably used for humans. Alternatively, the device according to the invention can also improve the relaxation state of animals, for example during the transportation of horses or dogs, so that no medication is necessary.

The device according to the invention provides an actuator 1 for actuating a brain stimulator 2 and a massager 3. The actuator 1 is formed as a mobile data apparatus, preferably as a mobile phone (smartphone), with generic operating, display, and control means. In the mobile data apparatus 1, a relaxation application is implemented with which an operating person can influence the apparatuses 2, 3 in an operatively simple manner. The mobile data apparatus 1 is coupled to the brain stimulator 2 and the massager 3 via a wireless communication interface such as bluetooth or WLAN.

The relaxation application can have several relaxation programs which an operating person can choose to start and cycle through. For example, the relaxation application can have a relaxation program A, the brain stimulation controller A1 and the massage actuation A2 of which are represented in FIG. 2. For example, the relaxation application can have a further relaxation program B, the brain stimulation controller B1 and the massage actuation B2 of which are represented in FIG. 3. Preferably, the relaxation application can have further relaxation programs.

Upon starting the relaxation program A, a stimulation actuation signal 4 is transmitted to the brain stimulator 2 and a massage actuation signal 5 is transmitted to the massager 3. The brain stimulator 2 can, for example, be formed as wirelessly or wiredly operated headphones with two hearing capsules 6, 6′ which are respectively arranged on one ear of the living being.

The massager 3 is preferably formed as a massage chair provided with massage means for massaging in particular the back of the living being.

The brain stimulator 2 obtains a stimulation signal 7 which circumvents the ear to directly affect a brain 8 of the living being. Through a massage signal 9, the massager 3 directly affects body parts 19 of the living being or of a person who preferably operates the actuator 1.

The stimulation actuation signal 4 is formed such that a stimulation signal in the infrasonic range of a first frequency f1 is generated at the first hearing capsule 6 and a different stimulation signal of a frequency f2 in the infrasonic range is generated at the second hearing capsule 6′. To form a beat, a beat frequency is formed at which the stimulation signal 7 is induced in the brain 8 as the difference between the first frequency f1 and the second frequency f2. Preferably, it is a pulsating stimulation signal 7. In the present exemplary embodiment, the one carrier frequency f1 can be 114 Hz and the second carrier frequency f2 can be 124 Hz, so that a beat frequency of 10 Hz is obtained for the stimulation signal 7. Preferably, a rhythmic stimulation occurs within the brain, wherein the frequencies of the stimulation signal 7 can be recorded using an electroencephalogram (EEG). The beat frequency can be in a range between 1 Hz and 30 Hz. Preferably, it remains constant throughout the relaxation process. Alternatively, the beat frequency can also vary within a relaxation program.

According to the relaxation program A according to FIG. 2, upon starting the relaxation program A, the brain stimulation controller A1 and the massage actuation A2 are preferably switched on simultaneously. In a first time interval 10 (relaxation phase), the stimulation frequency is 14 Hz to 30 Hz. For example, it can drop from a beta frequency range (14 Hz to 30 Hz) continuously through an alpha frequency range (7 Hz to 14 Hz) down into a theta frequency range (3 Hz to 7 Hz). After a duration of, for example, 8 min, the second time interval 11 (use phase) begins, in which the stimulation frequency is continuously in the theta frequency range. A lowest point T of a progression curve of the stimulation signal 7 is located within this use phase. In the use phase, the person is in a relaxed or sleepy state. After the end of the use phase after 7 min, the third time interval 12 (recovery phase) begins, wherein the stimulation frequency gradually rises from the theta frequency range into the beta frequency range. The person is now once again in an attentive and concentrated state, so that they can perform their desired task.

During these three time intervals 10, 11, 12, a massage of a back area of the person is continuously performed, wherein a corresponding massage signal 9 affects the person. A progression curve of the massage signal 9 is constant throughout the three time intervals 10, 11, 12.

Naturally, the time intervals 10, 11, 12 can also vary. They can, for example, be in a range between 4 min and 10 min.

After a second relaxation program B according to FIG. 3, this can be used to facilitate the falling asleep of the person. The actuation of the brain stimulator 2 in the first two time intervals 10, 11 is identical to the embodiment according to FIG. 2. Only the third time interval 12′ differs, wherein the frequency range in the third time interval 12′—as in the second time interval 11—is in the theta frequency range. This way, the person is further kept in the sleepy state, wherein the length of the third time interval 12′ is set such that, after the end thereof, the person is in the delta frequency range (1 Hz to 3 Hz deep sleep). The massage actuation signal 5 is chosen such that the massage signal 9 decreases linearly starting at the second time interval 11. This way, the massage intensity gradually or continuously decreases in the second and third time intervals 11, 12′.

According to an alternative embodiment of the invention, the massage actuation signals 5 can, alternatively or additionally, also be discrete to form bursts of power as massage signals 9 which are equal or different in length.

Preferably, the stimulation signal 7 is overlaid with a music signal, so that the person can perceive, for example, calming music which is conducive to overcoming stress or reaching the relaxation state.

According to an alternative embodiment of the invention which is not shown, the massager has a warming function, so that body parts of the person are warmed during the relaxation process or relaxation program. This leads to an additional relaxation of the muscle groups.

1. A device for increasing the well-being of a living being, comprising
a brain stimulator (2) adapted for generating a stimulation signal (7) in the infrasonic range, the stimulation signal (7) adapted to stimulate the brain (8) of the living being, and
a massager (3) adapted for generating a massage signal (9) for massaging body parts of the living being.

2. The device according to claim 1, wherein an actuator (1) is provided, the actuator adapted to transmit a stimulation actuation signal (4) to the brain stimulator (2) to generate the stimulation signal (7) for actuating the brain (8) and to transmit a massage actuation signal (5) to the massager (3) to generate the massage signal (9) for massaging body parts of the living being.

3. The device according to claim 1, wherein the actuator (1) is a mobile data apparatus which is connected to the brain stimulator (2) and the massager (3) via a wireless communication interface.

4. The device according to claim 3, wherein the mobile data apparatus is a mobile phone with a display unit, an operating unit, and a micro-controller.

5. The device claim 1, wherein a number of relaxation programs (A, B) are stored in the actuator (1), each having different brain stimulation controllers (A1, B1) and/or different massage actuations (A2, B2).

6. The device according to claim 5, wherein the brain stimulation controller (A1, B1) program is adapted to actuate the brain stimulator (2) to generate the stimulation signal (7) to form a beat frequency, wherein the beat frequency is the difference between two different carrier frequencies in the low-frequency infrasonic range.

7. The device according to claim 6, wherein the beat frequency is in a range 1 Hz to 30 Hz.

8. The device according to claim 1, wherein the stimulation signal (7) is overlaid with a music signal.

9. The device according to claim 1, wherein the brain stimulator (2) is adapted to be actuated such that
in a first time interval (10) of a relaxation process, the beat frequency of a frequency in the range from 14 Hz to 30 Hz is reduced,
in a second time interval (11), the beat frequency reaches a lowest point (T) in the range from 3 Hz to 7 Hz, and
in a third time interval (12), the beat frequency is increased to a frequency in the range of 14 Hz to 30 Hz or the frequency in the range between 3 Hz to 7 Hz is maintained.

10. The device according to claim 1, wherein the massager (3) is a massage chair.

11. A method for placing a living being in a relaxation state, comprising:
stimulating a brain (8) of the living being with a periodical stimulation signal (7) in an infrasonic range, characterized in that and,
at the same time, at least partially massaging a body of the living being using massage means.

12. The method according to claim 11, wherein a frequency of the stimulation signal (7) within a relaxation process gradually decreases and then increases again after passing a lowest point (T).

13. The method according to claim 11, wherein the progression of the frequency of the stimulation signal (7) is continuously changed.

14. The device according to claim 6, wherein the beat frequency is in the range 10 Hz to 30 Hz.

15. The device according to claim 1, wherein the massager (3) is a massage chair having back massage means.
